(19) United States
(12) Patent Application Publication
(10) Pub. No.: US 2003/0098022 A1
(43) Pub. Date: May 29, 2003

(54) Nebulizer optimal for patient at home care

(75) Inventors: Osamu Nakao, Kyoto-shi (JP); Koichi Tanaka, Kyoto-shi (JP); Tomoya Ishida, Kyoto-shi (JP); Koichi Takizawa, Kyoto-shi (JP)

Correspondence Address: Barry E. Bretschneider, Morrison & Foerster LLP, Suite 300, 1650 Tysons Boulevard, McLean, VA, 22102, US

(73) Assignee: Omron Corporation, Kyoto-shi (JP)

(21) Appl. No.: 10/303,892

(22) Filed: Nov. 26, 2002

(30) Foreign Application Priority Data
Nov. 27, 2001 (JP) 2001-361406

(51) Int. Cl. A61M 11/00

(52) U.S. Cl. 128/200.21; 600/530

(57) ABSTRACT

A nebulizer includes a peak flow meter measuring the respiratory function of a patient and a nebulizer as a inhaler of a liquid medicine. The patient blows in the breath from a peak flow meter blow-in section of the nebulizer into the peak flow meter to measure the respiratory function. Further, the patient inhales the liquid medicine in a liquid medicine bottle inserted into a liquid medicine bottle insert opening from a nebulizer inhale opening. Simultaneously, outer air measurement is performed with a temperature sensor and a humidity sensor of the nebulizer. Such measurement data and inhale recording data are transmitted to a server from an external connection. Thus, a health site of the patient based on the data is opened on a network. The patient can obtain appropriate advice from a doctor, and a liquid medicine supply from a service provider at appropriate timing.

### BACKGROUND OF THE INVENTION

1. Field of the Invention

The present invention relates to a nebulizer, and particularly, to a nebulizer capable of providing efficient and optimal service to a patient at home care.

2. Description of the Background Art

In diagnosis and treatment of respiratory diseases referred to as obstructive ventilatory impairments such as bronchitic asthma and chronic bronchitis, examination of respiratory function is generally performed using a respirometer (called as a spirometer) or the like, to provide treatment such as administration of medicines. In the examination of the respiratory function, the spirometer, or a peak flow meter that enables more simple and convenient examination, is used. In the administration of medicines, a therapy referred to as an aerosol inhale therapy, in which a medicine is nebulized into fine particles to be delivered directly into bronchial tubes in lungs, is becoming popular, and a nebulizer (an inhaler) employed in the therapy is coming into wide use.

On the other hand, a self medication, which means a self-management of health condition, home care and the like have been proposed recently, requesting patients to undergo self-management, such as to examine their own symptoms and take medicines accordingly. There has been a growing trend to use the above mentioned nebulizer as a tool for realizing such self-management. Additionally, network functions, such as the internet, connecting patients at home care and medical institutions has been spreading widely. Thus, the volume of information exchanged between patients and doctors is increasing. Furthermore, the range of home care is spreading.

As part of such home care, with improvements of recent network functions, Web pages in diary form are provided on the internet, to which a parent of a pediatric asthma patient can input treatment records and various data of the child, i.e., the patient, for transmitting to the doctor in charge. Such a Web page diary corresponds to conventional data which have been created by a doctor, inputting the contents of a diary brought by a patient into a computer. Such system reduces various works required conventionally, for example, the work for the doctor to input the contents, the work for the patient to record own treatment history or various data into a diary, and the work for the patient to bring the diary to submit the same to the doctor. Thus, the system is utilized in many ways, such as for providing the patients of treatment instructions, supporting the patients in acquiring information, storing the patients' data in an electronic database, and providing telemedicine. Accordingly, as many attempts are made to support home care, there is an increasing tendency to apply home care.

Though the conventional spirometer is an appropriate test equipment in determining the course of treatment for respiratory diseases in general, it is expensive for realizing such home care, and hence its wide use is hindered. Additionally, though a system for connecting to a computer to store test data or to effectuate search function is commercially available, it is difficult for the patient at home care to use, since it is designed for a special institution such as a medical institution.

Further, though the conventional peak flow meter is simple and convenient for the patient to measure his/her own condition to obtain necessary data for treatment, it is not provided with a data storage function. Thus, the patient must record the measurement value on his/her own every time the measurement is done. Additionally, the conventional peak flow meter is not provided with communication means to transmit the obtained measurement value to the medical institution, and therefore is not optimal for home care.

Still further, though the conventional nebulizers are widely available commercially in various types intended for home use, to be employed in home care, and are widely employed, it is difficult for the doctor to recognize specific treatment situation of the patient using the nebulizer. Thus, it is difficult for the doctor to provide detailed prescriptions and instructions when the condition of the patient changes. This is highly problematic especially when the patient at home care is a pediatric asthma patient. Specifically, in such a case, the doctor is required to obtain more detailed information, such as actual inhalation time period and amount of inhaled medicine on daily basis, health condition of the child, i.e., the patient, and environmental factors such as temperature, humidity and atmospheric pressure, and also required to address the matter even carefully. In such a situation, it is difficult to obtain each data as above with conventional nebulizer at home care, and hence a precise decision may be hindered.

The above mentioned temperature, humidity or atmospheric pressure of the environmental factors, are factors affecting symptoms of respiratory diseases, and thus their records are essential. Therefore, when using the conventional nebulizer at home care, the patient must read indications of a thermometer, a hygrometer, or a barometer and record them.

Additionally, since the conventional nebulizer is not provided with a communication function, it is difficult for the doctor to obtain real time information. Accordingly, when the type or amount of medicines that should be administered is wrong, it may not be recognized immediately. Further, since the conventional nebulizer is not provided with storage means, it is difficult for the doctor to obtain daily continuous data such as measurement data. Thus, it may be difficult to realize careful and efficient treatment.

In the conventional home care, it is the patient who purchases the nebulizer for use. On the other hand, the effectiveness of a medicine administration therapy is often determined in the course of the treatment, and hence, when the purchased nebulizer is determined to be not appropriate for the patient, it will only be wasted.

Further, basically, test result of respiratory function using the peak flow meter or the like, the type, dose and frequency or the like of the medicines to be administered from the nebulizer, must be associated with one another closely, while the conventional peak flow meter and nebulizer are separate devices and often used at different timings. Therefore, it is difficult for the doctor to recognize actual treatment situation efficiently, and thus the doctor is scarcely capable of giving instructions for multiple patients quickly.

Still further, when realizing the above mentioned home care using Web pages, it is necessary to precisely input data such as measurement results into a device with a communication function, such as a computer, which involves many problems such as mistakes in input or time required for input.

### SUMMARY OF THE INVENTION

Therefore, one object of the present invention is to provide a nebulizer, a server, a nebulizer system, a method of identifying a medicine in a nebulizer, a medicine amount detecting method in a nebulizer a method of managing information in nebulizer system, and a nebulizer information managing program product capable of providing efficient and optimal services to a patient at home care.

The above object of the present invention is achieved by a nebulizer including a treatment section for turning a medicine into nebulized particles and for spraying the same, and a measuring section for measuring respiratory function of a patient.

Preferably, the nebulizer further includes a display section for displaying, on one window, treatment record data from the treatment section and measurement result data from the measuring section.

Preferably, the nebulizer further includes an outer air measuring section for measuring at least one of temperature, humidity and atmospheric pressure.

Preferably, the nebulizer further includes a transmitting section for transmitting at least one of treatment record data from the treatment section, measurement result data from the measuring section, and outer air measurement result from the outer air measuring section, appending a patient identifier identifying the patient.

Preferably, the transmitting section transmits at least one of treatment record data from the treatment section, measurement result data from the measuring section, and outer air measurement result from the outer air measuring section appending a nebulizer identifier identifying the nebulizer.

According to another aspect of the present invention, a server includes a receiving section for receiving information from the transmitting section of the nebulizer, and a storage section for storing the received information.

Preferably, the server further includes an operating section for performing operating process on the stored information, and a window creating section for creating window data corresponding to at least one of the patient identifier identifying the patient and the nebulizer identifier identifying the nebulizer based on the stored information and the information performed with operating process.

According to still another aspect of the present invention, the nebulizer system includes the above server, a first processing device connected to the above nebulizer, a second processing device used by a doctor, and a third processing device used by a related service provider, connected with a network. The second processing device includes a transmitting section for transmitting a request for browsing the window specifying the patient identifier as a condition, and a password of the doctor requesting browsing of the window, a display section displaying the window when the password is authenticated at authenticating section of the server, and a transmitting section for transmitting a comment for the patient to the first processing device.

According to still another aspect of the present invention, the method of identifying a medicine in a nebulizer is characterized in that information of a medicine being encoded and attached to a medicine container is read by at least one of optical means and magnetic means.

According to still another aspect of the present invention, the medicine amount detecting method in a nebulizer is characterized in that medicine amount in a medicine container is detected by optical means.

According to still another aspect of the present invention, in the method of managing information in a nebulizer system including a server, a first processing device connected to a nebulizer, a second processing device used by a doctor, and a third processing device used by a related service provider, connected with a network, the second processing device executes a transmitting step of transmitting a request for browsing the window specifying the patient identifier as a condition, and a password of the doctor requesting browsing of the window, and a displaying step of displaying the window when the password is authenticated at authenticating step of the server, and a transmitting step of transmitting a comment for the patient to the first processing device.

According to still another aspect of the present invention, the nebulizer information managing program product causes a computer to execute a method of managing information in a nebulizer system including the server, a first processing device connected to the nebulizer, a second processing device used by a doctor, and a third processing device used by a related service provider, connected with a network, and causing the computer to execute in the second processing device a transmitting step of transmitting a request for browsing the window specifying the patient identifier as a condition, and a password of the doctor requesting browsing of the window, a displaying step of displaying the window when the password is authenticated at authenticating step of the server, and a transmitting step of transmitting a comment for the patient to the first processing device.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 shows one structure of a specific example of a nebulizer system in one embodiment according to the present invention;

FIG. 2 shows a specific example of the exterior of nebulizer 1 shown in FIG. 1;

FIG. 3 is a functional block diagram showing structure of nebulizer 1;

FIG. 4A shows a specific example of liquid medicine bottle 14;

FIG. 4B shows a specific example of liquid medicine identifying section 1091;

FIG. 4C shows a specific example of liquid medicine detecting section 1092;

FIG. 5 shows a specific example of peak flow meter section 110;

FIG. 6 shows a specific example of the structure of PC 3 shown in FIG. 1;

FIG. 7 shows a specific example of the structure of health server 5 shown in FIG. 1;

FIG. 8 shows a specific example of structure of doctor PC 7 shown in FIG. 1;

FIG. 9 shows a specific example of the structure of provider PC 9 shown in FIG. 1;

FIG. 10 is a flow chart showing with nebulizer 1;

FIG. 11 is a flow chart showing preparation process;

FIG. 12 is a flow chart showing inhaling process with nebulizer section 112;

FIG. 13 is a flow chart showing measuring process with peak flow meter section 110;

FIG. 14 shows a specific example of data transmitted to health server 5 from nebulizer 1;

FIG. 15 shows a data transmission process at step S225 in FIG. 12 using PC 3, with successively appearing windows on PC 3;

FIG. 16 shows a message sending and receiving process using PC 3, with successively appearing windows on PC 3;

FIG. 17 shows a health site browsing process using doctor PC 7, with successively appearing windows on doctor PC 7;

FIG. 18 shows a health site browsing process using provider PC 9, with successively appearing windows on provider PC 9;

FIGS. 19 and 20 show specific examples of windows of the health site;

FIG. 21 shows a specific example of the exterior of nebulizer 2;

FIG. 22 is a flow chart showing a renting and settling process with health server 5; and

FIG. 23 is a flow chart showing a user register process with PC 3.

### DESCRIPTION OF THE PREFERRED EMBODIMENTS

In the following, referring to the figures, embodiments of the present invention will be described. In the following description, the same parts and components are given similar reference characters. Their names and functions are also the same. Thus, detailed description thereof will not be repeated

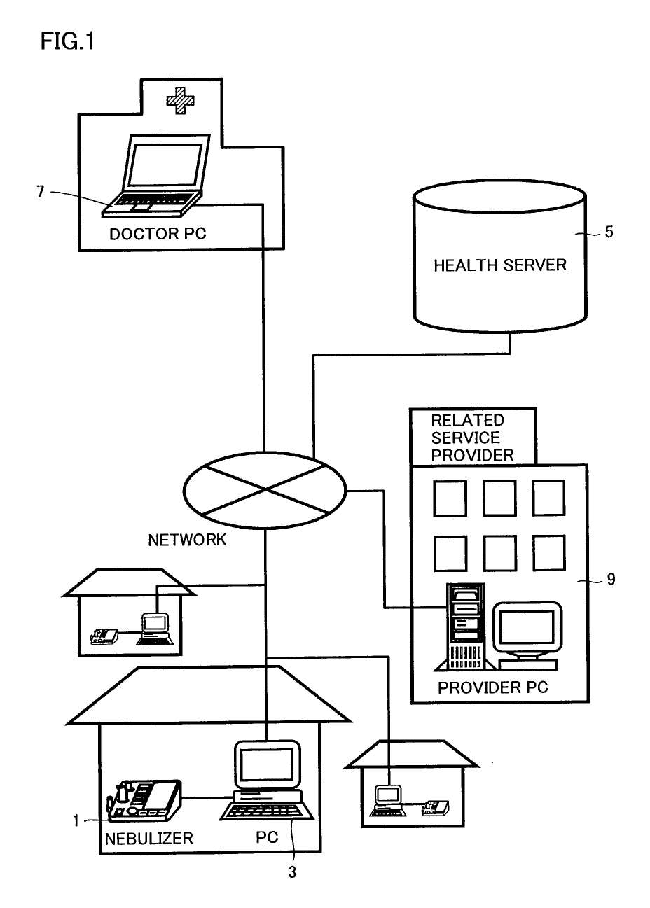

Referring to FIG. 1, a nebulizer system includes a nebulizer 1 provided at a patient's home or the like, a computer 3 (hereinafter referred to as PC) connected to nebulizer 1 for sending and receiving data, a server 5 for the health site (Web page) (hereinafter referred to as health server) centrally connected to PC 3 at each patient's home via network, a computer 7 installed at a hospital or the like for the doctor to browse data on health server and to give necessary instruction (hereinafter referred to as doctor PC), a computer 9 owned by a related service provider such as a pharmacy, connected to health server 5 via network (hereinafter referred to as provider PC).

In such a system, a user, i.e., the patient, executes prescribed process to rent nebulizer 1 from related service provider. The renting process will be described in due course.

The user will be at home care using rented nebulizer 1. The data generated by performing treatment using nebulizer 1 will be stored in health server 5 via network. A doctor or the like browses treatment data of the user stored in health server 5 on the health site, using doctor PC 7. Then, the doctor or the like may give instructions to the user by writing necessary instructions or the like on health server, or by sending an e-mail. Further, the doctor or the like by writing on health server liquid medicines that should be administered to the patient, then a pharmacy, i.e., the related service provider, browses the information on the health site and delivers the liquid medicines to the patient. Also, the related service provider utilizes health server 5 for processing settlement of the costs of renting nebulizer 1. The process related to renting nebulizer 1 will be described later.

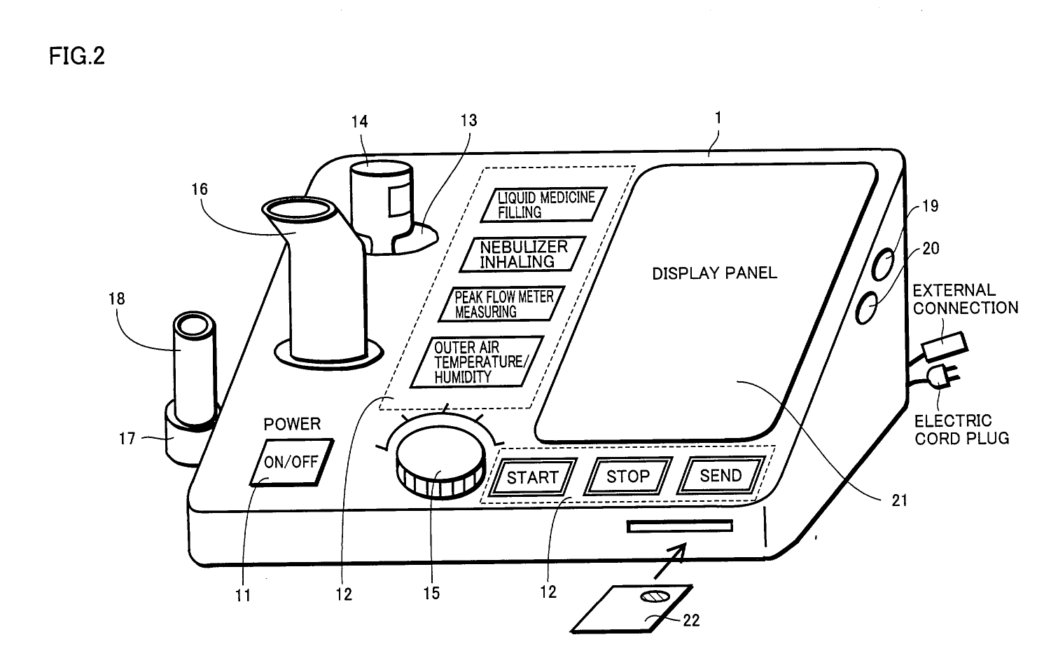

Nebulizer 1 shown in FIG. 2 according to the present invention is characterized in that it includes both a peak flow meter section for examining the user's lung ventilatory function, and a nebulizer section for inhaling liquid medicines. The peak flow meter section and the nebulizer section will be described in the description of nebulizer 1.

Referring to FIG. 2, nebulizer 1 includes a switch 11 for supplying power from power source, and function keys 12 for executing various processes.

Additionally, nebulizer 1 includes a liquid medicine bottle insert opening 13 for inserting a liquid medicine bottle 14 containing a respiratory liquid medicine, inhale timer 15, and nebulizer inhale opening 16 for administering nebulized liquid medicines. By putting nebulizer inhale opening 16 on the patient's mouth or nose, the liquid medicine nebulized into fine particles can be administered directly to bronchial tubes in lungs from nasal cavity or mouth cavity for a time period set by inhale timer 15.

Further, in nebulizer 1, a peak flow meter blow-in section 18 is provided to blow-in section stand 17. Peak flow meter blow-in section 18 is a cylindrical, so-called mouth-piece, and when the user, i.e., a patient, blows in a breath, senses lung ventilatory function of the patient.

Still further, nebulizer 1 accommodates a temperature sensor 19 and a humidity sensor 20, for recording temperature and humidity automatically when nebulizer 1 is used. Also, environmental temperature and humidity may automatically be recorded at prescribed intervals when nebulizer 1 is not used.

Still further, nebulizer 1 may accommodate atmospheric pressure sensor (not shown), for recording atmospheric pressure automatically when nebulizer 1 is used or not used.

Still further, nebulizer 1 includes a display panel 21 implemented by a liquid crystal panel or the like for displaying various information and measurement results.

In nebulizer 1, measurement results or treatment results are stored in the accommodated storage section and transmitted to health server 5 via network from external connection such as an interface, or they may also be recorded on a removable IC card 22. IC card 22 may be an IC card specific for nebulizer, or may be a so-called health care IC card that is an IC card for storing and managing information related to health care generally. In the latter case, not only measurement results and treatment results from nebulizer 1 will be recorded on IC card 22, but also information such as the user's clinical history, treatment history or the number of health insurance may be recorded for general use at a medical institution or the like.

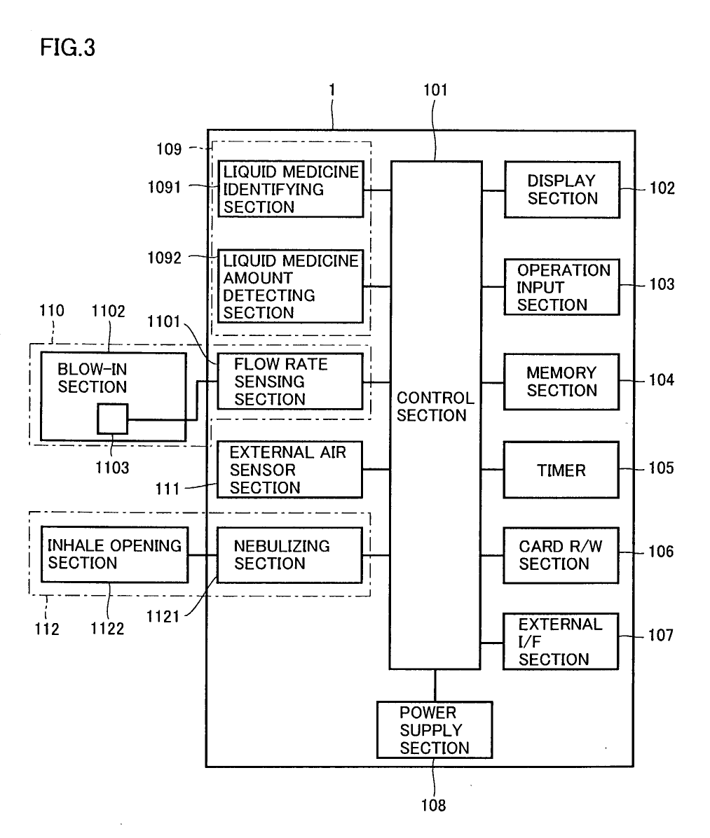

Next, referring to FIG. 3, the structure of nebulizer 1 will be described.

Referring to FIG. 3, nebulizer 1 is implemented with a power supply section 108 for supplying power to nebulizer 1, a control section 101 implemented with CPU (Central Processing Unit) or the like for performing overall control of nebulizer 1, a display section 102 implemented with display panel 21 or the like, an operation input section 103 implemented with function keys 12 or the like, a memory section 104 for storing programs executed by control section 101 as well as various measurement results and also for serving as a work area when programs are executed by control section 101, a timer 105 implemented with blow-in timer 15 or the like, a card R/W (read/write) section 106 for reading and writing information to and from IC card 22, and an external I/F (interface) section 107 for connecting to PC 3 or the like.

Nebulizer 1 further includes a liquid medicine bottle insert opening 109 implemented by a liquid medicine identifying section 1091 and a liquid medicine amount detecting section 1092; a peak flow meter section 110 implemented by blow-in section 1102 including a rotary-vane tachometer section 1103 and a flow rate sensing section 1101; an external air sensor section 111 implemented by temperature sensor 19, humidity sensor 20 and pressure sensor which is not shown; and a nebulizer section 112 implemented by a nebulizing section 1121 and an inhale opening section 1122.

Nebulizing section 1121 of nebulizer section 112 nebulizes the liquid medicine, filled in liquid medicine bottle 14 inserted in liquid medicine bottle inserting opening 109, using ultrasonic wave. The process at nebulizing section 1121 will not be described here in detail. The nebulized liquid medicines at nebulizing section 1121 will be sprayed from inhale opening 1122.

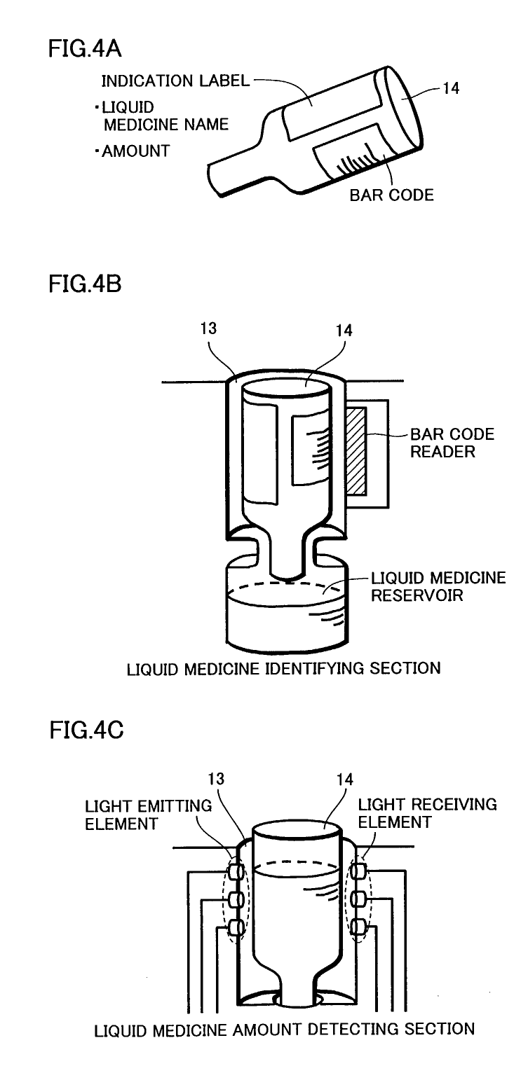

Referring to FIG. 4A, liquid medicine bottle 14 is provided with indication label showing information such as the name of the liquid medicine, and bar codes recording such information.

Next, FIG. 4B shows liquid medicine identifying section 1091 of liquid medicine bottle insert opening 109. Referring to FIG. 4B, liquid medicine identifying section 1091 includes a bar code reader implemented by LED (Light Emitting Diode) and a light receiving line sensor, for optically reading information such as the name of the liquid medicine from the bar codes on the above mentioned liquid medicine bottle 14. Note that liquid medicine determining method at liquid medicine identifying section 1091 is not limited to optical reading and determining method, and it may be magnetic reading and determining method. Other determination method may be employed.

FIG. 4C shows liquid medicine amount detecting section 1092 of liquid medicine bottle insert opening 109. Referring to FIG. 4C, liquid medicine amount detecting section 1092 is provided with prescribed numbers of light emitting elements and light receiving elements arranged vertically. The amount of light transmitting through the bottle at the liquid medicine containing portion and at the remaining portion (empty portion) after emitted from the light emitting element, are different depending on the difference of index of refraction. By the light receiving elements that are provided corresponding to the light emitting elements receive transmitted lights deferring in amount, the position of the liquid medicine in the bottle can be determined, and hence, the amount of the liquid medicine in liquid medicine bottle 14 can be detected. Note that, the liquid medicine amount detecting method at liquid medicine amount detecting section 1092 is not limited to the optical detecting method above, and may be other detecting method.

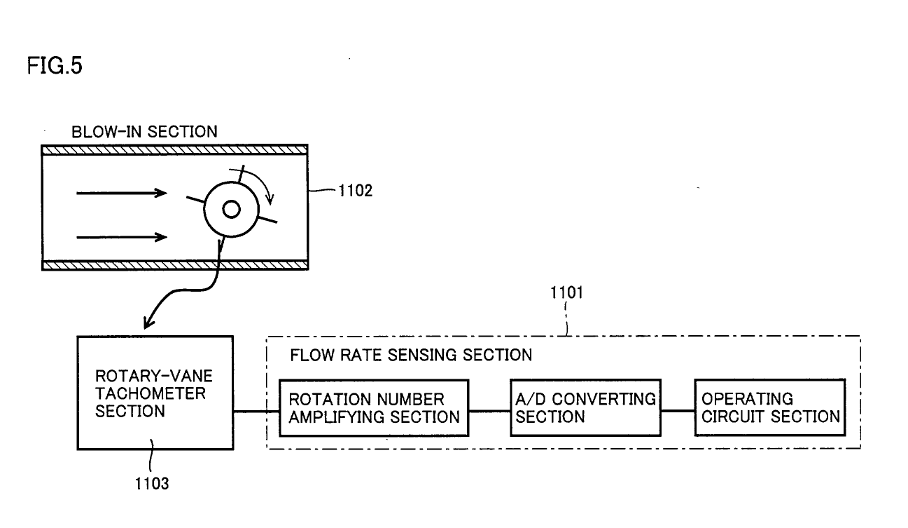

FIG. 5 shows a specific example of peak flow meter section 110. Peak flow meter section 110 measures PEF (Peak Expiratory Flow) value, which is the maximum exhale flow rate (liter/minute) forced out from lungs after taking a deep breath. By the PEF value, the width of airway of the user i.e., a patient, can be determined. Specifically, if the PEF value is greater than a prescribed threshold value, then the airway is wide, and if the value is smaller than the prescribed threshold value, then the airway is narrow. Accordingly, the PEF value is the measurement value which plays an important role in recognizing the lung ventilatory function of the user.

Preferably, the user may check the lung ventilatory function with peak flow meter section 110, before inhaling the liquid medicine using nebulizer 1. Further, in view of self medication, in order to check his/her own health condition on usual basis, it is preferable for the patient to measure lung ventilatory function by peak flow meter section 110 regularly.

Referring to FIG. 5, blow-in section 1102 of peak flow meter section 110, implemented by peak flow meter blow-in section 18, includes a rotary-vane tachometer inside. A rotary-vane tachometer section 1103 implemented by the rotary-vane tachometer above transmits the value of rotary-vane tachometer, which rotates corresponding to exhaled output of the user's breath coming in from peak flow meter blown-in section 18, to flow rate sensing section 1101. Flow rate sensing section 1101 is implemented by a rotation number amplifying section, an A/D converting section and an operating circuit section, and measures the user's lung ventilatory function by turning the value sent from rotary-vane tachometer section 1103 into the PEF value. Note that the method of measuring user's exhale flow rate with peak flow meter section 110 is not limited to the measuring method above, and it may be a measuring method using pressure sensor, a strain gauge or the like.

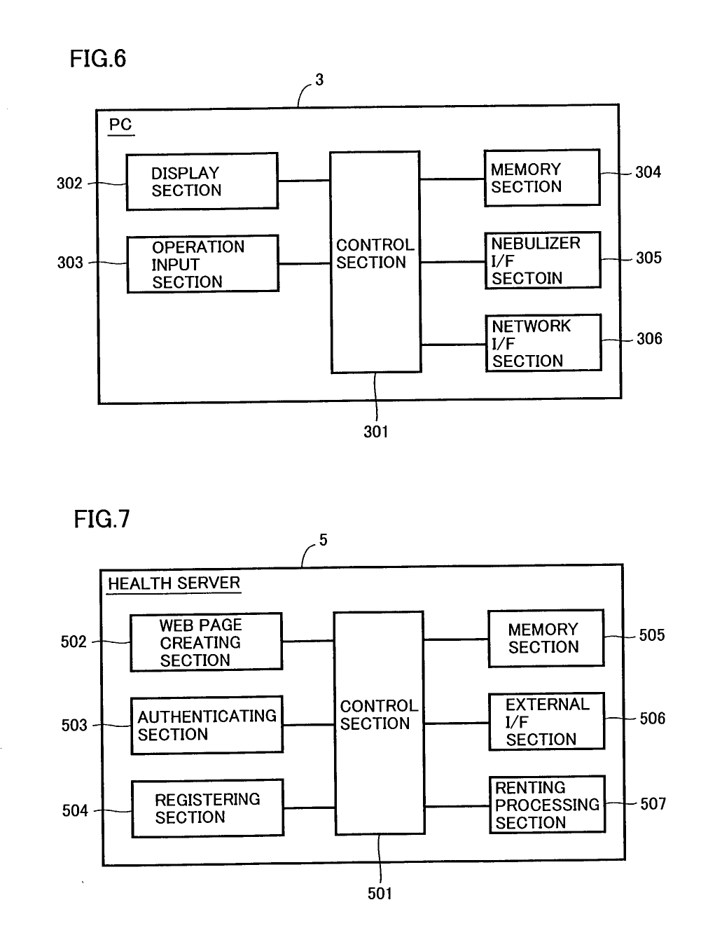

Next, FIG. 6 illustrates a specific example of structure of PC 3 shown in FIG. 1.

Referring to FIG. 6, PC 3 includes a control section 301 implemented with CPU or like and performing overall control of PC 3, a display section 302 implemented with a display or the like for displaying various information, an operation input section 303 implemented with keyboard or the like for performing various operation or input of instructions or the like, a memory section 304 for storing programs or the like executed by control section 301 and also for serving as work area when the program is executed by control section 301, a nebulizer I/F section 305 connected to nebulizer 1 for sending and receiving various information, and a network I/F section 306 connecting to network and sending and receiving various information to/from health server 5 or the like.

Note that the structure of PC 3 shown in FIG. 6 is a structure of a computer in general, and it is not limited to the structure shown. Further, in the FIG. 1, though PC 3 is included in the nebulizer system and to be a personal computer as a computer connecting to nebulizer 1 for sending and receiving data, PC 3 is not limited to a computer and may be a mobile communication terminal such as a cellular phone with similar function.

Next, FIG. 7 shows a specific example of the structure of health server 5 shown in FIG. 1.

Referring to FIG. 7, health server 5 includes control section 501 implemented with CPU or the like for performing overall control of health server 5, an external I/F section 506 connected to network for sending and receiving various information to and from PC 3 or the like, a memory section 505 for storing data obtained from nebulizer 1 via external I/F section 506 and programs executed by control section 501, and for serving as a work area when programs are executed by control section 501, a Web page creating section 502 for creating and updating the health site opened on the network according to data stored in memory section 505, an authenticating section 503 for authenticating a user accessing the health server 5, a registering section 504 for recording a user accessible to health server 5, and renting processing section 507 for performing processes related to renting of nebulizer 1 in nebulizer system.

Note that the structure of health server 5 shown in FIG. 7 is the structure of server in general, and thus it can be implemented by using personal computer or the like. Additionally, the structure of health server 5 is not limited to that shown in FIG. 7.

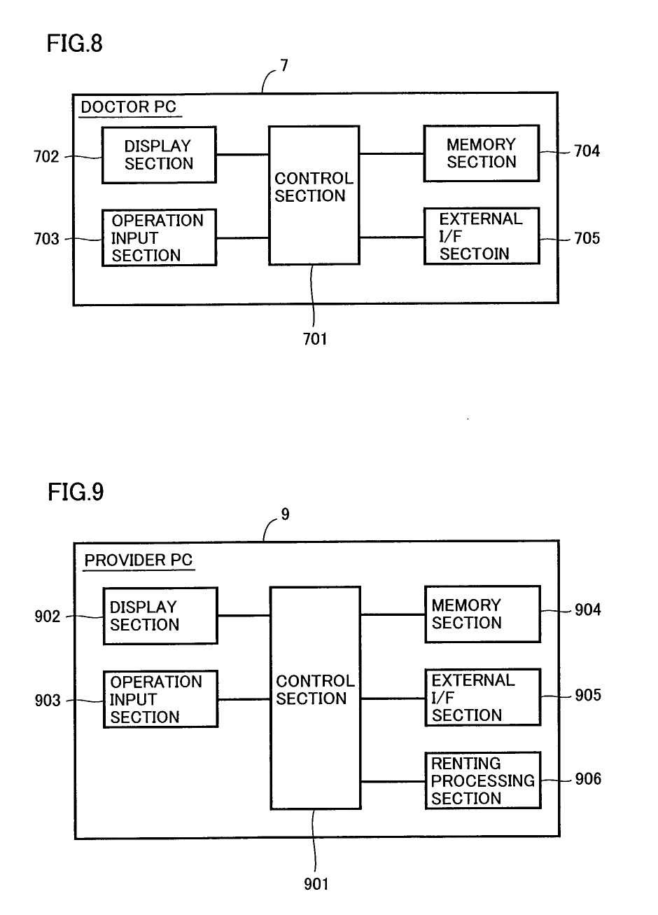

Next, FIG. 8 shows a specific example of doctor PC 7 shown in FIG. 1.

Referring to FIG. 8, doctor PC 7 includes control section 701 implemented with CPU or the like for performing overall control of doctor PC 7, a display section 702 implemented with a display or the like for displaying various information, an operation input section 703 implemented with a keyboard or the like for performing various operations and input of instructions or the like, a memory section 704 for storing programs executed by control section 701 and for serving as a work area when the programs are executed by control section 701, and an external I/F section 705 connected to network for sending and receiving various information to and from health server 5 or the like.

Note that the structure of doctor PC 7 shown in FIG. 8 is the structure of computer in general, and it is not limited to the illustrated structure.

Next, FIG. 9 shows a specific example of the structure of provider PC 9 shown in FIG. 1.

Referring to FIG. 9, provider PC 9 includes a control section 901 implemented with CPU or the like for performing overall control of provider PC 9, a display section 902 implemented with a display or the like for displaying various information, an operation input section 903 implemented with a keyboard or the like for performing various operations and input of instructions or the like, a memory section 904 for storing programs executed by control section 901 and for serving as a work area when the programs are executed by control section 901, an external I/F section 905 connected to network for sending and receiving various information to and from health server 5 or the like, and a renting processing section 906 for performing processes related to renting of nebulizer 1 in the nebulizer system.

Note that the structure of provider PC 9 shown in FIG. 9 is the structure of a computer in general, and it is not limited to the shown structure.

In the following, description will be given about a treatment process which is executed in the nebulizer system stated above.

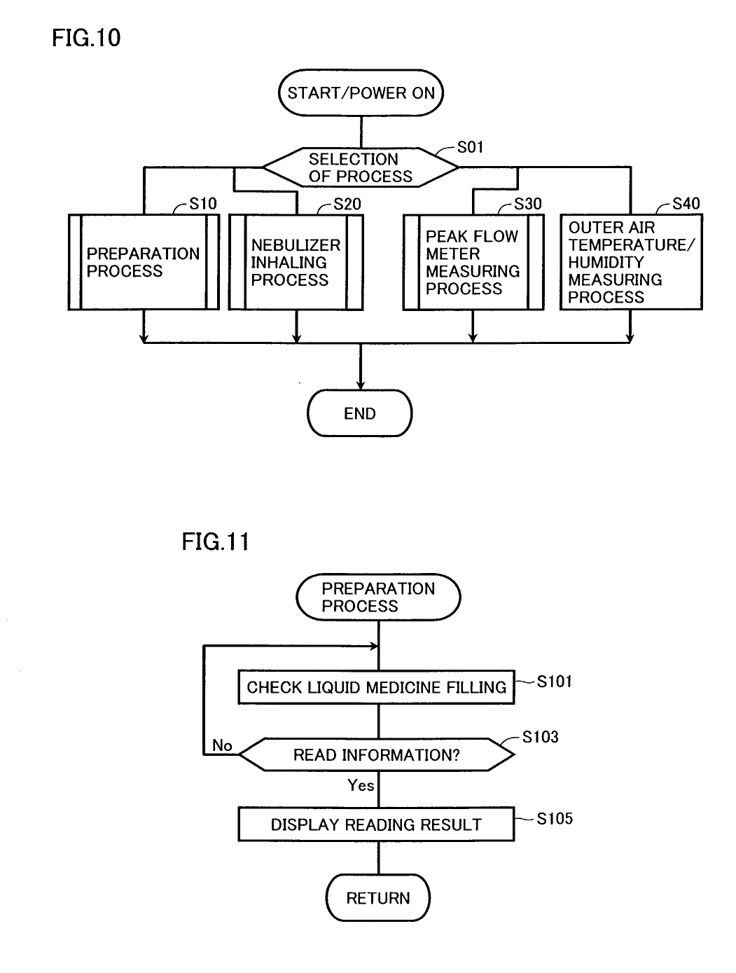

FIG. 10 is a flow chart showing the process at nebulizer 1. The process shown in FIG. 10 is realized by control section 101 of nebulizer 1 executing programs stored in memory section 104.

Referring to FIG. 10, nebulizer 1 initiates the program when the switch 11 is pressed and power supply section 108 supplies power.

Subsequently, if a user (a patient) inputs the selection of the process from operation input section 103 of function keys 12 or the like (S01), then control section 101 of nebulizer 1 executes any one of the following steps: the liquid medicine preparation process (S10), the inhaling process with nebulizer section 112 (S20), the exhale flow rate measuring process with peak flow meter section 110 (S30), and the outer air temperature and humidity measuring process with outer air sensing section 111 (S40).

The outer air temperature and humidity measuring process shown at step S40 is the process executed by the user pressing down the outer air temperature and humidity measuring key of function keys 12, so as to cause outer air sensor section 111 implemented with temperature sensor 19, humidity sensor 20 and the like to measure the outer air temperature and humidity, for storing the data in the prescribed area of memory section 104. The measurement value may be obtained through single measurement, or may be obtained through prescribed numbers of measurements followed by an operating process for determining the averaged value. Additionally, the outer air measuring process may be performed automatically in prescribed intervals. Further, when outer air sensor section 111 is provided with an atmospheric pressure sensor (not shown), the atmospheric pressure is measured and stored.

For the processes shown in S10 to S30, description will be given in the following with a subroutine.

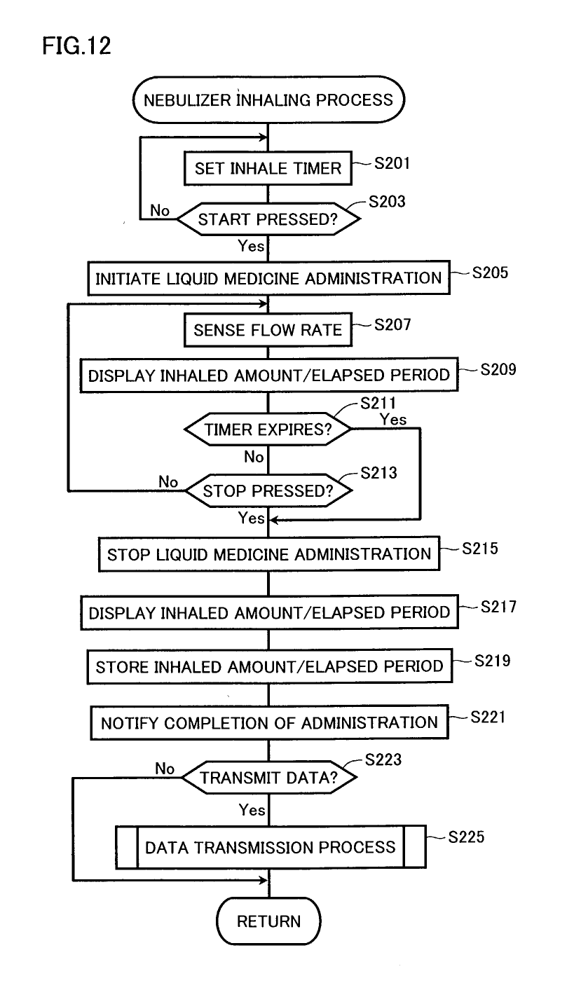

FIG. 11 is a flow chart showing a preparation process. The preparation process shown in FIG. 11 is a subroutine executed at step S10 in FIG. 10, and it is initiated by a user pressing down liquid medicine filling key of function keys 12 at step S01 in FIG. 10.

Referring to FIG. 11, when a user presses the liquid medicine filling key of function keys 12, nebulizer 1 checks at liquid medicine bottle insert opening 109 whether the liquid medicine is filled (S101). Specifically, control section 101 checks whether the liquid medicine is filled by causing liquid medicine identifying section 1091 to read the liquid medicine name or the like of liquid medicine bottle 14 inserted in liquid medicine bottle insert opening 13, and by causing liquid medicine amount detection section 1092 to read liquid medicine amount (S101).

If the above information can not be read at liquid medicine bottle insert opening 109 (No at S103), then there is a possibility that liquid medicine bottle 14 is not inserted in liquid medicine bottle insert opening 13. In such a case, control section 101 may perform some processes, such as to instruct display section 102 to indicate the above situation on a display panel 21, and again checks if liquid medicine is filled at step S101.

If the above information is read at liquid medicine bottle insert opening 109 (Yes at S103), then control section 101 instructs display panel 102 to display the information of liquid medicine name or the like read at step S101 on a display panel 21 (S105).

Thus the preparation process shown in step S10 in FIG. 10 ends, and the process returns to the main routine.

Next, FIG. 12 is a flow chart showing inhale process at nebulizer section 112. The inhale process shown in FIG. 12 is a subroutine executed at step S20 of FIG. 10, and it is initiated by the user pressing nebulizer inhale key of function keys 12 at step S01 in FIG. 10. Note that the inhale process shown in the step S20 in FIG. 10 is executed only when preparation process shown in step S10 is completed. Accordingly, when it is selected in step 01 that the inhale process of S20 is to be performed, the system may check if the preparation process of step S10 is completed, and if the preparation process is not completed, then it may display the situation and terminate the inhale process. In the following, description will be given assuming that the preparation process is completed at step S10.

Referring to FIG. 12, when the user presses the nebulizer inhale key of function keys 12, control section 101 receives the setting of inhale period from inhale timer 15, and sets the input period into timer 105 (S201).

Thereafter, if the user presses down start key of function keys 12 (Yes at S203), control section 101 instructs nebulizer section 112 to administer the liquid medicine (S205). Simultaneously, control section 101 measures administered amount (S207), and instruct display section 102 to display the amount inhaled by the user and the elapsed time on display panel 21 (S209). At step S207, the administered liquid medicine amount may be calculated from the amount of the liquid medicine contained in the liquid medicine bottle 14 which is detected by liquid medicine amount detection section 1092, or when nebulizer section 112 is provided with a flow meter, which is not shown, it may be measured by the flow meter.

If the timer period set at step S201 is expired (Yes at S211), or a stop key of function keys 12 is pressed before the expiration of the timer period (Yes at S213), then control section 101 instructs nebulizer section 112 to stop the liquid medicine administration process above (S215), and further instructs display section 102 to display the total amount inhaled and inhale time period by the user on display panel 21 (S217). Still further, the total inhaled amount and the inhale time period by the user are stored in a prescribed area of memory section 104 (S219).

Thereafter, control section 101 instructs display section 102 to display the completion of liquid medicine administration on display panel 21 to notify the user (S221).

If the user presses a transmission key of function keys 12 to select the process of sending the data of total inhaled and inhale time period by the user to health server 5 (Yes at S223), then control section 101 sends the data to health server 5 from external I/F section 107 via network (S225). Note that, at step S225, the transmission process is realized by connecting external I/F section 107 of nebulizer 1 to nebulizer I/F section 305 of PC 3, and executing the program by CPU 301 of PC 3. The data transmission process executed at PC 3 will be described in detail in due course referring to a subroutine.

If the user does not send the data (No at S223), the inhaling process shown in step S20 of FIG. 10 ends and the process is returned to the main routine.

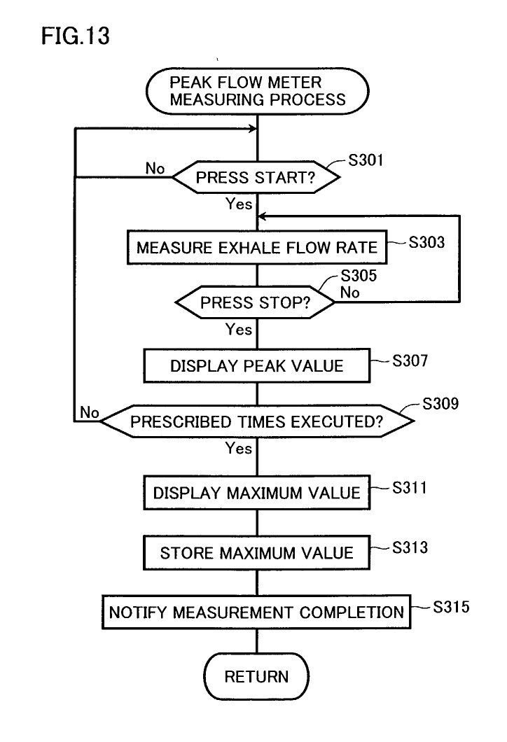

Next, FIG. 13 is a flow chart showing a measurement process at peak flow meter section 110. The measuring process shown in FIG. 13 is a subroutine executed at step S30 of FIG. 10, and is initiated by the user pressing down peak flow meter measuring key of function keys 12. It is preferred that the measuring process shown at step S30 in FIG. 10 is executed prior to the inhaling process shown at step S20. Thus, the user can preferably comprehend the condition of the lung ventilatory function to perform the inhaling process corresponding to the condition. Therefore, if it is selected at step S01 that the nebulizer inhale process of step S20 should be performed, then control section 101 may check if the measuring process of step S30 is completed, and if the measuring process is not completed, control section 101 may instruct to display the situation to prompt the execution of the measuring process.

Referring to FIG. 13, if the user presses peak flow meter measuring key of function keys 12, and further presses start key (Yes at S301), peak flow meter section 110 continuously measures exhale flow rate of the user (S303) until stop key is pressed (Yes at S305).

If stop key is pressed to complete measurement of above exhale flow rate, then control section 101 instructs display section 102 to display the PEF value, which is the maximum exhale flow rate, on display panel 21 (S307).

If processes at the above steps S301 to 307 are executed for preset times (S309), and prescribed times of the processes are completed, then control section 101 instructs display section 102 to display the highest measured value in the prescribed numbers of measurements on display panel 21 (S311), and further stores the value in the prescribed area of memory section 104. Note that the measurement value displayed and stored at steps S311 and S313 may not be limited to the highest value, but on the contrary it may be the average value of maximum exhale flow rate of each measurement, and it may be the other value.

Thereafter, control section 101 instructs display section 102 to display completion of measurement on display panel 21 for notifying the same to the user (S315).

Thus, the measurement process shown at step S30 in FIG. 10 is completed, and the process is returned to the main routine.

The four processes (S10 to S40) shown in FIG. 10 constitute the main routine of the processes executed with nebulizer 1 in the present embodiment, and with the completion of the above processes, the program at nebulizer 1 ends. Usually, nebulizer 1 executes steps in order of steps S10, 30, and 20. Specifically, firstly the liquid medicine is filled to complete the treatment preparation at nebulizer 1, and subsequently nebulizer 1 measures the PEF value of the user with peak flow meter section 110. Thereafter, nebulizer 1 provides treatment (inhaling process) using nebulizer section 112. Further, nebulizer 1 simultaneously measures the outer air temperature and humidity as shown at step S40. The measurement results and the treatment results in the sequence of processes will be displayed on display panel 21 by the instruction to display section 102 after completion of each process. And at completion of the sequence of processes, the measurement results and the treatment results are all displayed on display panel 21. Thus, the user can check the measurement results and the treatment results by display panel 21 at one time after executing the sequence of the processes.

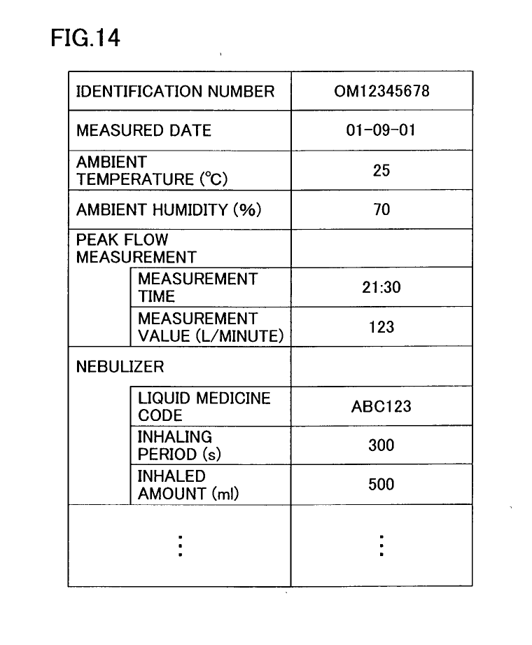

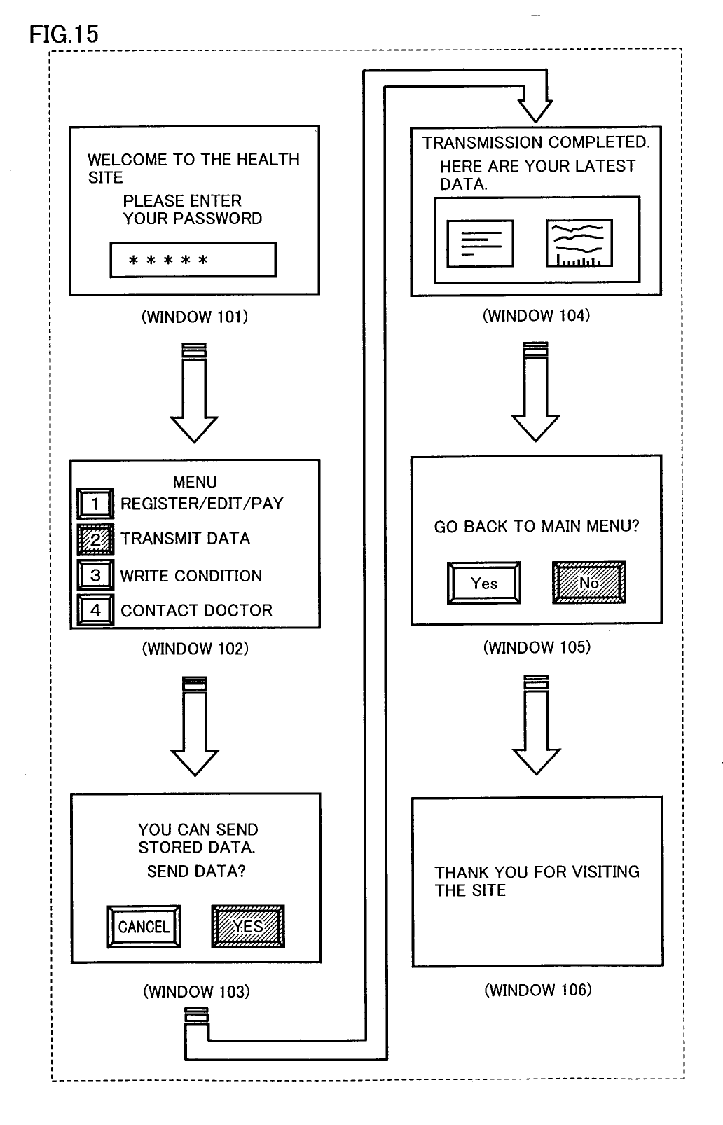

A specific example of data transmitted from nebulizer 1 to health server 5 is shown in FIG. 14. The data for transmission shown in FIG. 14 are measured in the process of steps S20 to S40 of FIG. 10, and are generated based on the data stored in a prescribed area of memory section 104. Note that the data may be generated in nebulizer 1 or may be generated in the connected PC 3.

Referring to FIG. 14, the data to be transmitted includes identification number information identifying nebulizer 1, measurement date information, outer air measurement result information with outer air sensor section 111, measurement result information with peak flow meter section 110, and treatment information with nebulizer section 112.

The above identification number is specific to nebulizer 1. The identification number of nebulizer 1 is recorded on health server 5 when the user registers his or her use at health server 5, associated with the user's (patient's) name and password. Health server 5 utilizes the identification number to manage each user's data. Note that the identification number is an identifier for identifying the terminal, and it may be other information other than numbers.

Further, in FIG. 15, the data transmission process shown at step S225 of FIG. 12 using PC 3 is shown by successively appearing windows on the display of PC 3. The windows (windows 101 to 106) shown in FIG. 15 are windows displayed on display section 302 showing the progress of programs, as control section 301 of PC 3 executes the programs stored in memory section 304.

Referring to FIG. 15, at step S225 in FIG. 12, the user transmitting the measurement data or the like accesses health server 5 using PC 3 via network. At that time, window 101 is displayed on display section 302.

Window 101 is for the user to perform a log-in process. First, PC 3 accepts the log-in process by the user, on window 101. Specifically, PC 3 accepts input of password pre-registered on health server 5. At this time, authenticating section 503 of health server 5 accepting the password of the user via network, refers to authentication information stored in memory section 505, to perform authentication of the user. If the user's authentication succeeds at health server 5, then window 102 will appear on the display.

Window 102 is a menu displaying window. PC 3 accepts the data transmission process as a selected result by the user from the menu shown on window 102. If the data transmission process is selected, PC 3 takes data for transmission from memory section 104 of nebulizer 1 connected to PC 3 via external I/F section 107 and nebulizer I/F section 305. When the transmission of the data is completed, window 103 will be displayed. If corresponding data are not stored at this time, or any other event occurs, the situation may be displayed on a window to notify the user and the process may be terminated.

Window 103 is a data transmission window. If PC 3 accepts the data transmission instruction by the user following dialogs on window 103, then sends data for transmission shown in FIG. 14 to health server 5 from network I/F section 306 via network. The transmitted data are received at health server 5 via external I/F section 506, and stored in prescribed area of memory section 505. Further, Web page creating section 502 of health server 5 updates the user's the health site based on the received data. And, as the transmission is completed, window 104 will be displayed on display section 302 of PC 3.

Window 104 is a window notifying completion of the data transmission. Window 104 notifies the user that data has been transmitted. Further, window 104 may notify the user of the data of the user's the health site which are updated in health server 5. Thereafter, window 105 will appear on the display.

Window 105 is for the user to give an instructions of whether going back to menu window shown on window 102 or not. If PC 3 receives an instruction from the user to go back to the menu window, then window 102 will again be displayed on display section 302. If PC 3 receives an instruction from the user not to go back to the menu window, then a window 106 for notifying completion of the transmission process is displayed.

Thus, the data transmission process is completed.

Note that, the user can send and receive message to and from the doctor, or can write daily condition into the health site by selecting from the menu window shown on window 102 in FIG. 15. In the following, specific examples thereof will be given and will be described.

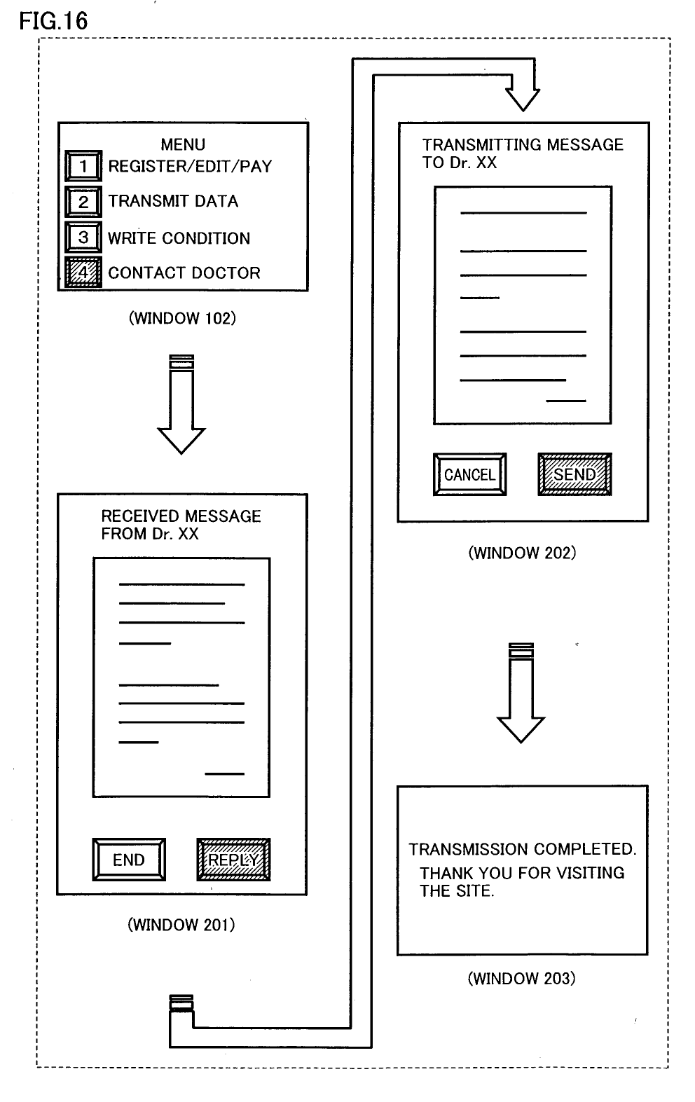

If PC 3 accepts a process of contacting doctor as a result of selection from menu window shown on window 102 in FIG. 15, then it executes a process shown in FIG. 16. FIG. 16 shows a message exchanging process using PC 3, with windows successively appearing on the display of PC 3. The windows shown in the FIG. 16 (window 102, 201 to 203) are also the windows displayed along the progress of the program, stored in memory section 304 and executed by control section 301 of PC 3.

Referring to FIG. 16, if PC 3 accepts a process for contacting doctor from the user as a result of selection in the process shown on window 102 of FIG. 15, PC 3 reads prescribed area of memory section 505 of health server 5 to check the presence of a message from the doctor. Then, if the message from the doctor is found, then window 201 will be displayed.

Window 201 displays a message from the doctor. By referring to window 201, the user can obtain an advice or an instruction from the doctor who has viewed the user's the health site. PC 3 reads advises or the like from the doctor from prescribed area of memory section 505 of health server 5 and displays on window 201 via network and external I/F section 506.

Additionally, the user can also send a reply to the doctor. When input of reply key by the user is accepted on window 201, window 202 will appear on the display.

Window 202 is for the user to send a reply. When input of transmission key by the user on window 202 is accepted, PC 3 sends the input information to health server 5 from network I/F section 306 via network. Health server 5 receiving the information via external I/F section 506 stores the same in a prescribed area of memory section 505. Thereafter, when the transmission to health server 5 is completed, window 203 for notifying the user of completion of transmission process is displayed.

Thus, the message exchanging process is completed.

Accordingly, the measured data or the like which have been transmitted to health server 5 from each user (patient) are stored in a prescribed area of memory section 505 of health server 5. Further, Web page creating section 502 of health server 5 creates each user's the health site based on the measurement data. The created the health site is opened on a network, and the user, the doctor, and the related service provider and the like authorized by passwords can browse the site.

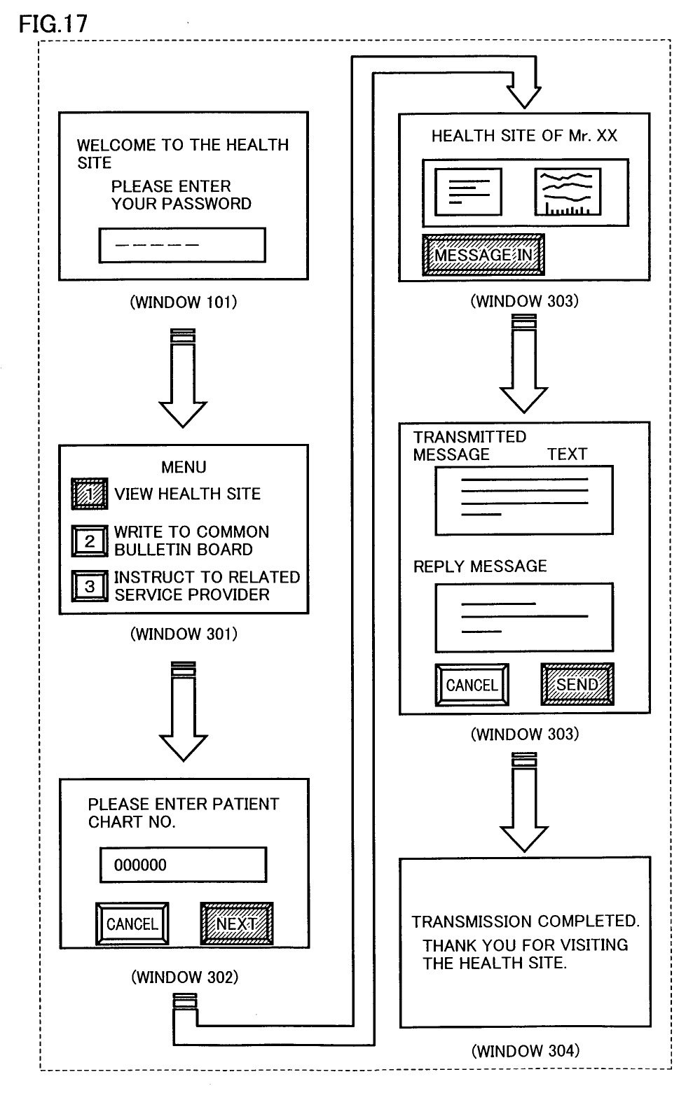

Subsequently, a process for browsing the health site for the doctor or the like using doctor PC 7 will be described referring to FIG. 17. FIG. 17 illustrates a health site browsing process using doctor PC 7, with successively appearing windows on doctor PC 7. Window displays shown in FIG. 17 (windows 101, 301 to 304) are the windows displayed on display section 702 along the progress of the program, stored in memory section 704 and executed by control section 701 of doctor PC 7.

Referring to FIG. 17, the doctor, who is to browse the health site, accesses health server 5 via network using doctor PC 7. At this time, a window similar to window 101 shown in FIG. 15 will be displayed on display section 702.

Window 101 is for the doctor to execute a log-in process. First, doctor PC 7 accepts the log-in process from the doctor on window 101. Specifically, doctor PC 7 accepts input of the password pre-registered at health server 5. At this time, authenticating section 503 of health server 5 accepting doctor's password via network refers to authentication information stored in memory section 505 to authorize the doctor. If authentication of the doctor at health server 5 succeeds, then window 301 will appear on the display.

Window 301 is a menu displaying window. Doctor PC 7 accepts browsing of the health site process as a result of selection from the menu shown on window 301 from the doctor. Then, window 302 will appear on the display.

Window 302 will appear when doctor PC 7 accepts input of the patient's (user's) chart number of the health site of which the doctor wishes to browse. The chart number is an identification number allocated to the user when the doctor sees the user for the first time (on the first visit), and is an identifier stored along with the data of the user in health server 5. The chart number being input into doctor PC 7 is transmitted to health server 5 from external I/F section 705 via network. The transmitted chart number is received at health server 5 via external I/F section 506. Health server 5 reads information of the user of interest from the prescribed area of memory section 505 based on the chart number being received. Then, window 303 will appear on the display.

Window 303 is a window of the user's health site. The health site shown on window 303 will be described in due course with a specific example. By viewing window 303, the doctor can check the user's health condition for providing appropriate diagnosis or the like. Additionally, window 303 displays a message, such as a question, from the user to the doctor. If the doctor views the message, the doctor will input his intention into doctor PC 7, on window 303. When the instruction is accepted, doctor PC 7 reads from external I/F section 705 a message of interest from the prescribed area of memory section 505 of health server 5 via network and external I/F section 506. Then, window 304 will appear on the display.

Window 304 is for the exchange of messages between the doctor and the user. Window 304 displays message from the user to the doctor. Further, doctor PC 7 accepts reply to the message. As the doctor inputs reply and presses the transmission key, the input information into doctor PC 7 will be sent from external I/F section 705 to health server 5 via network and external I/F section 506 to be stored in a prescribed area of memory section 505. Then, when the data transmission is completed, window 304 for notifying the doctor of the transmission process completion will appear on display section 702 of doctor PC 7.

Thus, the health site browsing process will be completed.

Note that, the doctor can use the menu displaying window shown on window 301 of FIG. 17 to perform a write process to a common bulletin board or to perform an instruction process to related service providers, via health server 5. The common bulletin board is a bulletin board shared by a network of patient having common symptoms or a network of doctors of the same field, for accepting general questions by the patients or for providing appropriate advice from the doctors, or for promoting communication between patients. Additionally, instructions to the related service provider, such as a new prescription of liquid medicine for the user or update information of nebulizer 1, may be given via health server 5.

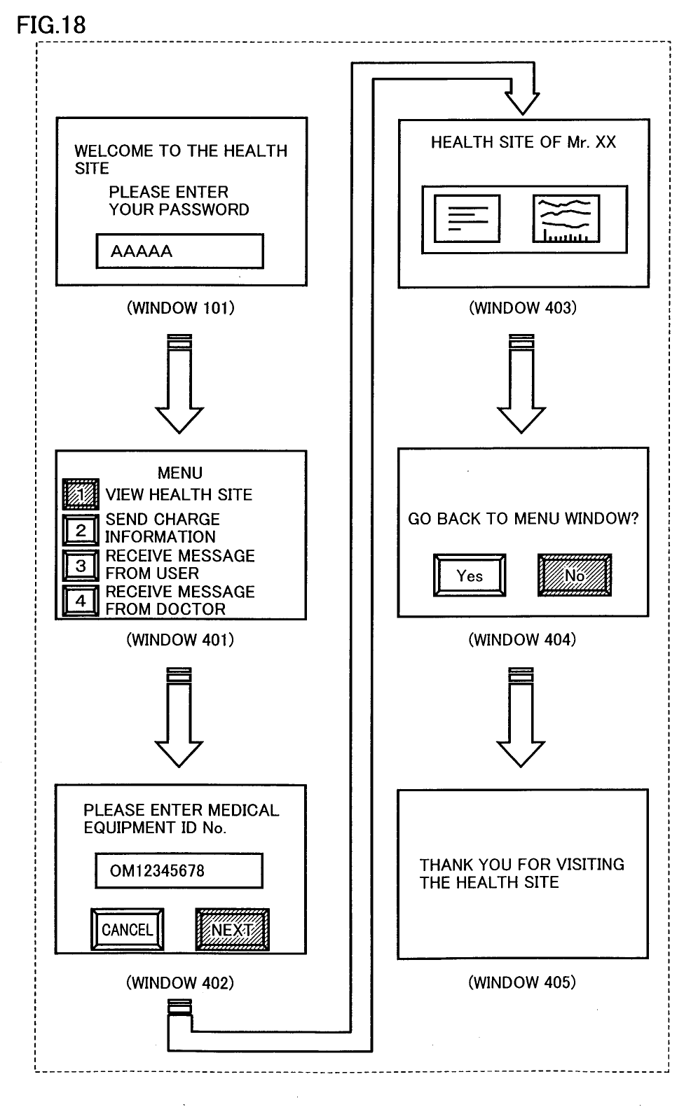

Subsequently, the health site browsing process for the related service provider using provider PC 9 will be described referring to FIG. 18. FIG. 18 shows a health site browsing process using provider PC 9, with successively appearing windows on provider PC 9. Window displays shown in FIG. 18 (windows 101, 401 to 405) will appear along the progress of a program on display section 902, which has been stored in memory section 904 and is executed by control section 901 of provider PC 9.

Referring to FIG. 18, the related service provider, in order to view the health site, accesses health server 5 via network using provider PC 9. At that time, a window similar to window 101 shown in FIG. 15 will appear on display section 902.

Window 101 is for the related service provider to perform a log-in process. First, provider PC 9 accepts the log-in process from the related service provider on window 101. Specifically, provider PC 9 accepts input of the password pre-registered in health server 5. At this time, authenticating section 503 of health server 5 accepting the password of related service provider via network refers authentication information stored in memory section 505 to authenticate the related service provider. If authentication of the related service provider succeeds at health server 5, then window 401 will appear on the display.

Window 401 is a menu displaying window. Provider PC 9 accepts a health site browsing process as a result of selection from the menu shown on window 401 by the related service provider. Then, window 402 will appear on the display.

Window 402 is a window appears when provider PC 9 accepts an input of identification number of medical equipment such as nebulizer 1 rented to the patient (user) of the health site to be viewed. The identification number being input into provider PC 9 is transmitted to health server 5 from external I/F section 905 via network. The transmitted identification number will be received at health server 5 via external I/F section 506. Health server 5 reads information of the user, based on the received identification number, who is using medical equipment such as nebulizer 1 and corresponds to the identification number, from prescribed area of the memory section 505. Then, window 403 will appear on the display.

Window 403 is a window of the user's the health site. The health site shown on window 403 will be described in due course with a specific example. By viewing window 403, the related service provider can check user's health condition or diagnosis from a doctor, as well as type or remaining amount of the liquid medicine administered to the user. Therefore, the related service provider can prepare for orders of users in advance. Then, window 404 will appear on the display.

Window 404 is for the related service provider to give instructions whether going back to the menu window shown on window 401. If provider PC 9 accepts an instruction from the related service provider to go back to the menu window, then window 401 will be displayed again on display section 902. If provider PC 9 accepts an instruction from the related service provider not to go back to menu window, then window 405 for notifying completion of the process will be displayed.

Thus, the health site browsing process will be completed.

Note that, in the above mentioned window 401, by selecting the message receiving process from the user or the message receiving process from the doctor, the related service provider can receive a message from the user or an instruction of prescription from the doctor via health server 5, and respond to it quickly. Also, by selecting the charge information transmission process, the related service provider can adjust costs associated with renting of medical equipment, such as nebulizer 1, via health server 5.

Next, the health site opened by health server 5 on a network will be described.

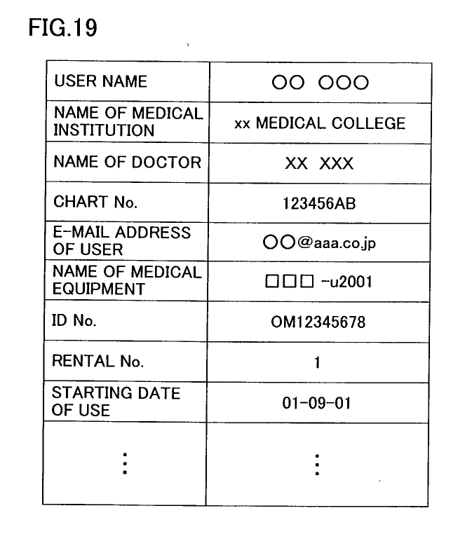

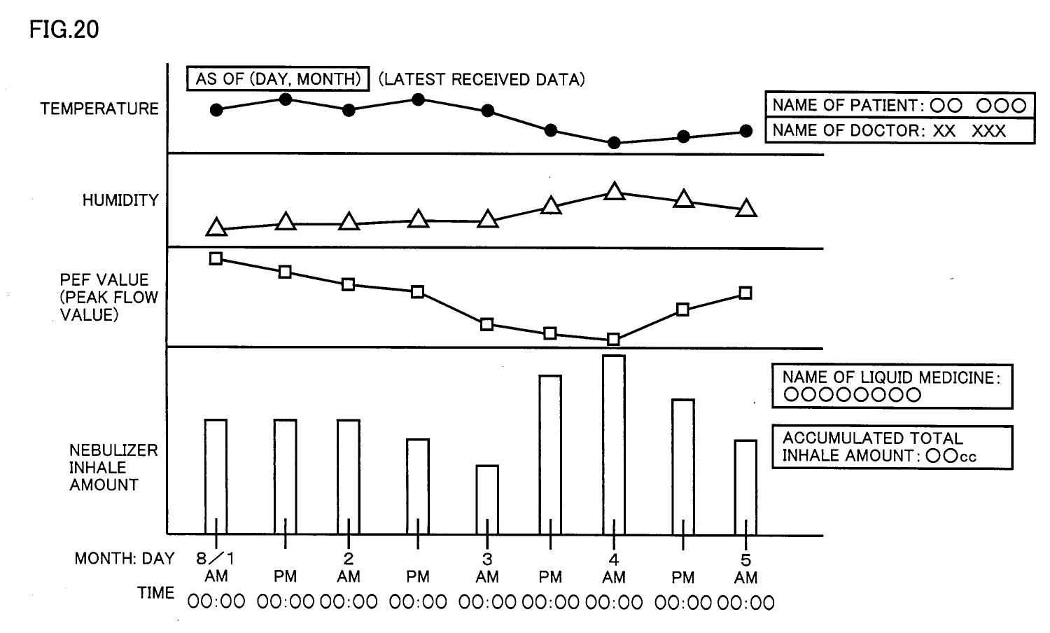

FIGS. 19 and 20 show specific example of a window of the health site. The specific example shown in FIGS. 19 and 20 may appear together in one window of the health site, or may be separate windows respectively displayed based on selection in the menu window which is not shown. Web page creating section 502 performs various statistical processes and modifications to the data, stored in a prescribed area of memory section 505 of health server 5 and transmitted from each user's PC 3. Specifically, processes such as calculating the averaged value or turning the data into graph are performed. The health site shown in FIGS. 19 and 20 is created for each user by Web page creating section 502 of health server 5 and opened on the network, based on transmitted data from each user's PC 3 thus processed with statistical process as above, user record information stored in memory section 505 of health server 5, and various information transmitted from doctor PC 7 of doctor.

First, in the health site shown in FIG. 19, the user's information is displayed. Specifically, information such as user name, the name of medical institution, the name of doctor in charge, chart number, e-mail address of the user, name of medical equipment such as nebulizer 1, identification number of the medical equipment, the rental number of the medical equipment, and the starting date of using the medical equipment. Note that, among the information displayed in the health site shown in FIG. 19, the name of the medical equipment, the identification number of the medical equipment, the rental number of the medical equipment, and the starting date of using the medical equipment will be automatically read from nebulizer 1 when the user sends data measured by nebulizer 1 using PC 3 to health server 5, and will be automatically registered at health server 5. Other information includes information input by the user performing user registration or information input by doctors or the like.

Next, on the health site shown in FIG. 20, information based on measured data of nebulizer 1 is displayed. Specifically, information such as date of giving treatment, temperature and humidity, the PEF value of peak flow meter, type of the liquid medicine being administered, inhaled amount of the liquid medicine calculated by inhaling period, and total accumulating amount of the inhaled liquid medicine will be displayed in graphs. In the specific example shown in FIG. 20, displayed information is based on measure data obtained by a user performing the treatment twice a day, i.e., once in the morning and once in the afternoon. As shown in FIG. 20, such information may be integrally calculated and displayed for predetermined items in Web page creating section 502 of health server 5, of which results will be stored in the prescribed area of memory section 505. Then, they may appear on a window following instruction of the user, the doctor and the like. Additionally, modification on the data or the like, not shown in the FIG. 20, may be performed by instructions from the user, the doctor and the like. Further, as described referring to FIGS. 16 and 17, messages can be exchanged between the user and the doctor or the like on the health site.

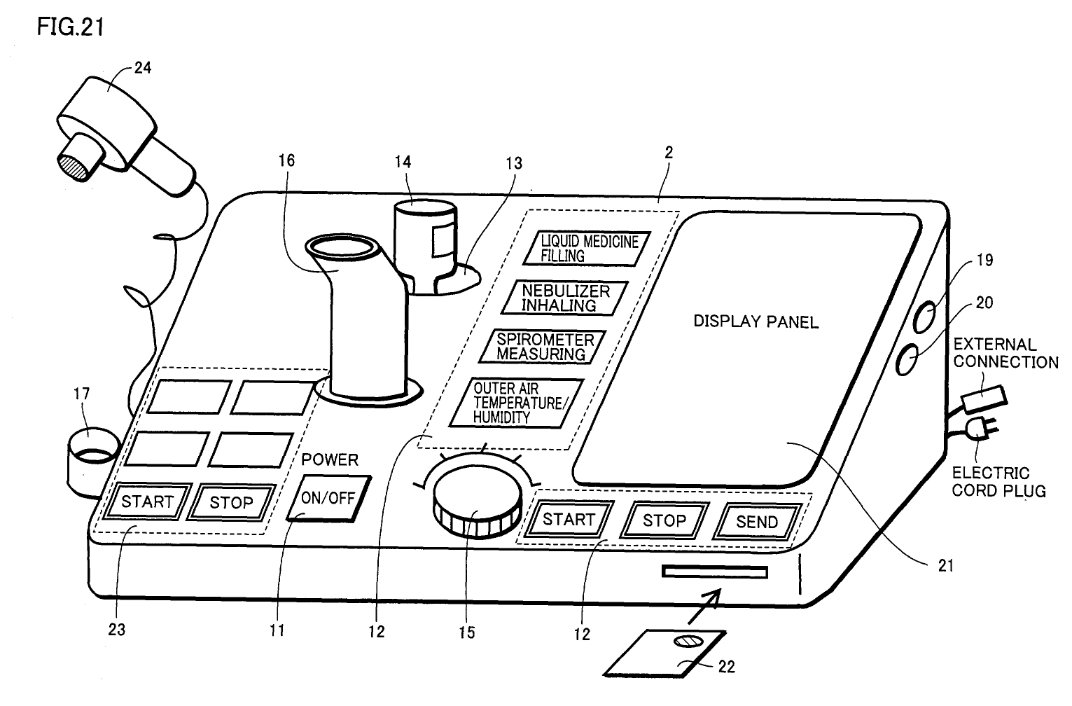

Note that, though the specific example of the window of the health site shown in FIGS. 19 and 20 illustrate the user's the health site using nebulizer 1 of FIG. 2 for treatment, a nebulizer included in the nebulizer system is not limited to nebulizer 1 shown in FIG. 2. Accordingly, as another specific example, a specific example for nebulizer 2 will be shown in FIG. 21.

Nebulizer 2 shown in FIG. 21 is characterized in including a spirometer section in place of peak flow meter of nebulizer 1 shown in FIG. 2, and thus including both of the spirometer section and the nebulizer section.

The spirometer is a device for examining lung ventilatory function, and examines the user's lung ventilatory function by measuring the user's vital capacity, ventilation amount and the like. Since such measuring methods are popular, detailed description thereof will not be given. In the present embodiment, necessary data for treatment are obtained by measuring, specifically, the vital capacity and ratio of the forced expiratory volume in one second, using the spirometer. The vital capacity is also referred to as forced vital capacity, which is determined by measuring vital capacity while taking a deep breath and then exhaling as fast as possible. The ratio of the forced expiratory volume in one second may be determined by measuring vital capacity in the first one second when exhaling as fast as possible, and then calculating the ratio of the measured value relative to forced vital capacity. Dropped ratio of the forced expiratory volume in one second may indicates obstructive impairments of lungs such as bronchial asthma.

Referring to FIG. 21, nebulizer 2 includes a spirometer blow-in section 24 which is a so called mouth-piece, in place of peak flow meter blow-in section 18 of nebulizer 1 shown in FIG. 2. Additionally, it includes a spirometer function keys 23 for operating the spirometer section.

After pressing start key of spirometer function keys 23, the user puts spirometer blow-in section 24 on the mouth, and then exhales breath making sure that the breath is not leaked from the nose. After blowing out all the breath, the user presses stop key of spirometer function keys 23. Thus, the user's lung ventilatory function is measured with the spirometer section.

Preferably, the user checks the lung ventilatory function by the spirometer section prior to having treatment with the nebulizer section of nebulizer 2. Further, from a viewpoint of self medication, the user preferably measures the lung ventilatory function by spirometer section regularly, in order to be aware of own health condition on usual basis.

The user's lung ventilatory function measurement data, obtained by the spirometer section of nebulizer 2 as above, will be treated similarly as the measurement data obtained by nebulizer 1 as described above, in the nebulizer system. Specifically, based on the measurement data obtained by nebulizer 2, health server 5 creates the user's the health site shown in FIGS. 19 and 20, and opens the same on the network.

As above, in the nebulizer system according to the present embodiment, an identification number is allocated to medical equipment such as nebulizer 1, and further a chart number is allocated to the user using nebulizer 1. Health server 5 stores the user's information, i.e., a patient's information, based on the above identification number of medical equipment and chart number. Therefore, the doctor or the like can browse the user's the health site on the network shown in FIGS. 19 and 20 based on chart number of the user, to obtain all of the user's treatment data including the environmental data such as temperature and humidity. Additionally, the change of user's health condition can be comprehended at a glance. Further, the information can be checked at real time. Still further, these data can be checked any time since these data are stored on health server 5. Hence, the doctor can provide better diagnosis as well as detailed and efficient treatment.

Still further, the related service provider, pre-registered on the nebulizer system according to the present embodiment, can browse the user's the health site who is using the nebulizer 1, based on the above identification number of medical equipment shown in FIGS. 19 and 20, by its own provider PC 9. The related service provider can check the type or remaining amount of the liquid medicine administered to the user by referring to the health site shown in FIGS. 19 and 20, and following the instruction of the doctor or based on a contract settled in advance, the related service provider can further provide services such as supplying the liquid medicine to the user before run out. Therefore, the user at home care may continue to have treatment at home without concern for the remaining amount of the liquid medicine and without the trouble of ordering the liquid medicine. Further, the related service provider can grasp the treatment condition of the patient using nebulizer 1, based on the terminal number of nebulizer 1, and thus, the related service provider can not only expect the liquid medicine to be supplied, but can provide consumable items of nebulizer 1, address troubles of nebulizer 1 quickly, check quality of nebulizer 1, update the version of nebulizer 1, or provide other items utilized in treatment. Thus, the related service provider can provide services ahead of the user's need.

Such nebulizer system can be a system for supporting efficient home care, and may contribute greatly to home care of the user, i.e., a patient, and to the self medication of the user.

Since the nebulizer network includes the system of the related service provider (provider PC 9), not only appropriate treatment process is executed, but also a renting and settling process of medical equipment such as nebulizer can be executed.

In the following, the renting and settling process executed with the above nebulizer system will be described.

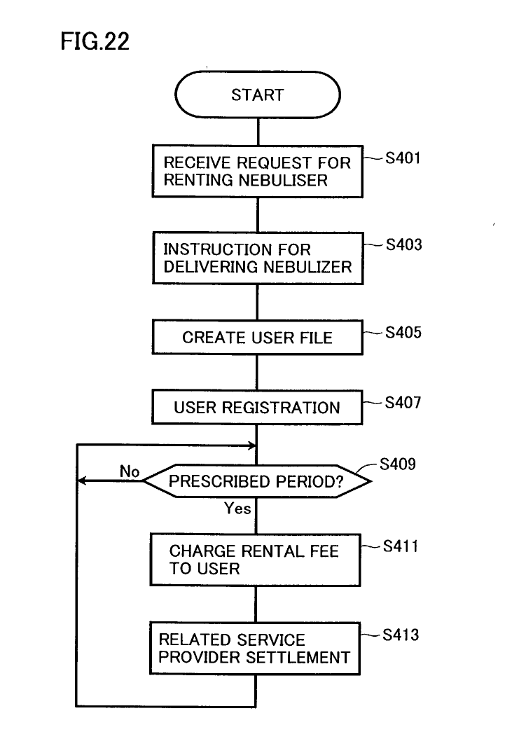

FIG. 22 is a flow chart showing the renting and settling process in health server 5. The process in FIG. 22 is realized by executing a program stored in memory section 505 by control section 501 of health server 5.

Referring to FIG. 22, if external I/F section 506 of health server 5 receives a message requesting renting of nebulizer 1 from user's PC 3 via network (S401), health server 5 transmits an instruction of delivering nebulizer 1 to provider PC 9 of the related service provider renting nebulizer 1, from external I/F section 506 via network (S403).

Simultaneously, registration section 504 of health server 5 uses the identification number of nebulizer 1, of which delivery has been instructed to provider PC 9 at step S403, to create a file for storing various information of the user in the prescribed area of memory section 505 (S405). The contents of the file created at step S405 is similar to that of the user information displayed on the health site shown in FIG. 19.

Then, if external I/F section 506 receives registration to an nebulizer system from the user being delivered of nebulizer 1 by the related service provider (S407), then control section 501 of health server 5 determines that the use of nebulizer 1 is initiated. User registration process to nebulizer system using PC 3 will be described in due course.

Control section 501 of health server 5 stores a use period of nebulizer 1 in the prescribed area of memory section 505 for each identification number of nebulizer 1. Then, when the use period reaches a prescribed period (Yes at S409), renting processing section 507 calculates the rental fee, and health server 5 transmits the charge of rental fee of nebulizer 1 to user's PC 3 from external I/F section 506 via network (S411). Charging of rental fee transmitted to PC 3 at step S411 may be done by sending a message using e-mail or the like, or it may be done by other method. Further, at step S407, if a settlement to credit company or the like is registered and also a system owned by the credit company is included in the nebulizer system, then health server 5 may transmit the charge of rental fee to the system owned by the credit company.

Additionally, health server 5 executes electronic settlement of costs including rental fee of nebulizer system 1 to renting processing section 906 of provider PC 9, from external I/F section 506 via network and external I/F section 705 (S413).

Processes at steps S411 and S413 may be repeated at prescribed timings, such as at the end of every month.

By repeating the above program, the renting and settling process is executed in health server 5, for the user of nebulizer 1 included in the nebulizer system, and for the related service provider.

In the following, the user registration performed by the user with PC 3 at the above step S407 will be described.

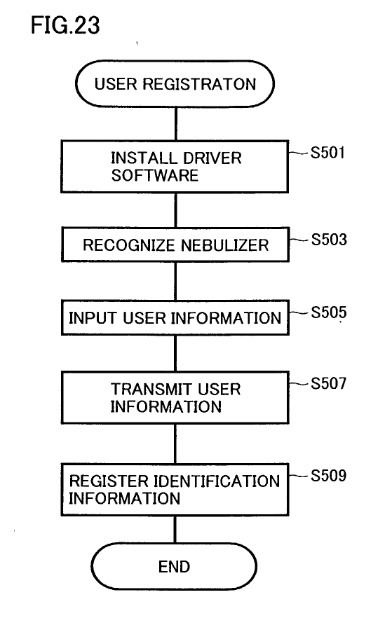

FIG. 23 is a flow chart showing user registration process in PC 3. The process shown in FIG. 23 is realized by executing a program stored in memory section 304 by control section 301 of PC 3.

Referring to FIG. 23, in order to perform the registration process, firstly driver software delivered with nebulizer 1 is installed (S501). At step S501, if the driver software is installed, then nebulizer I/F section 305 will recognize nebulizer 1 as connected (S503).

Subsequently, on a window for inputting information displayed on display section 302, PC 3 accepts input of prescribed user information by the user (S505). Further, PC 3 transmits accepted user information to health server 5 from network I/F section 306 via network (S507).

An identification number of nebulizer 1 is attached to the user information being sent. At health server 5 receiving these user information at external I/F section 506 via network, registration section 504 extract the user's file created in the prescribed area of memory section 505 at step S405 in FIG. 22 based on the identification number attached to user's information. Then, registration section 504 adds received user information to the extracted user's file.

Further, the user's PC 3 sets ID, i.e., an identifiers of the user, and password for health server 5 (S509).

Thus, the process of user registration to nebulizer system for the user of nebulizer 1 is completed.

As a possible related service provider of the nebulizer system, a distribution center managing inventory, or a pharmacy managing liquid medicines may be included. In such a case, health server 5 may send a nebulizer delivery instruction to the distribution center at step S403 in FIG. 22, and may settle the delivery cost at step S413. Additionally, a process of prescribing and delivering the liquid medicine, not shown in FIG. 22, is also possible, and in which case health server 5 accepts prescription of liquid medicines for the user i.e., a patient, from doctor PC 7, and then gives the pharmacy a prescription instruction and a delivery to the user instruction, and further executes the settlement process for the user and the pharmacy.

As described above, in the nebulizer system according to the present embodiment, by allocating the identification number to medical equipment such as nebulizer 1 and managing the medical equipment such as nebulizer 1 based on the identification number at health server 5, the renting and settling process can also be executed in a unified way. As a result, more efficient system for supporting home care can be established, further contributing to home care and self medication of the user, i.e., a patient.

It should be noted that, though in the present embodiment the description is given for the nebulizer system providing treatment, for symptoms associated with lung function such as asthma, using nebulizer shown in FIGS. 2 and 21 as medical equipment, the medical equipment to be included in the system is not limited to the nebulizer, and other medical equipment can be utilized to provide treatment related to other symptoms.

Additionally, in the above nebulizer system, it is stated that the user transmits data obtained by nebulizer 1 to health server 5 using PC 3, but the patient may also transmit data to health server 5 by writing the data into IC card 22 and then bring IC card 22 to a hospital or the like to have the card read by an installed card reader or the like.

Further, the treatment method, the renting and settlement method executed in the nebulizer system described above can be provided as programs. Such programs are computer readable programs. The programs may be recorded on recording media, such as flexible disc attached to a computer, CD-ROM (Compact Disc-Read Only Memory), ROM, RAM (Random Access Memory) and memory card, and may be provided as program products. The programs may be provided by recording on the recording media such as hard disc included in a computer. Further, the programs may be provided by downloading via network. The program products provided may be installed in the program storage such as hard disc to be executed. It should be noted that the program products include the programs themselves and the recording media recorded with the programs.

Although the present invention has been described and illustrated in detail, it is clearly understood that the same is by way of illustration and example only and is not to be taken by way of limitation, the spirit and scope of the present invention being limited only by the terms of the appended claims.

What is claimed is:

1. A nebulizer, comprising:
a treatment section for turning a medicine into nebulized particles and for spraying the particles; and
a measuring section for measuring respiratory function of a patient.

2. The nebulizer according to claim 1, further comprising
a display section for displaying, on one window, treatment record data from said treatment section and measurement result data from said measuring section.

3. The nebulizer according to claim 1, further comprising
an outer air measuring section for measuring at least one of temperature, humidity and atmospheric pressure.

4. The nebulizer according to claim 1, further comprising
a memory section for storing at least one of treatment record data from said treatment section, measurement result data from said measuring section, and outer air measurement result data from outer air measuring section measuring at least one of temperature, humidity and atmospheric pressure.

5. The nebulizer according to claim 4, wherein
said memory section appends a patient identifier identifying said patient to at least one of treatment record data from said treatment section, measurement result data from said measuring section, and outer air measurement result data from said outer air measuring section and stores the data.

6. The nebulizer according to claim 4, wherein
said memory section stores data with a memory device detachable from said nebulizer.

7. The nebulizer according to claim 6, wherein
said memory device is an IC card.

8. The nebulizer according to claim 1, further comprising
a transmitting section for transmitting at least one of treatment record data from said treatment section, measurement result data from said measuring section, and outer air measurement result data from said outer air measuring section measuring at least one of temperature, humidity and atmospheric pressure.

9. The nebulizer according to claim 8, wherein
said transmitting section appends a patient identifier identifying said patient to at least one of treatment record data from said treatment section, measurement result data from said measuring section, and outer air measurement result data from said outer air measuring section and transmits the data.

10. The nebulizer according to claim 8, wherein
said transmitting section appends a nebulizer identifier identifying the nebulizer to at least one of treatment record data from said treatment section, measurement result data from said measuring section, and outer air measurement result data from said outer air measuring section and transmits the data.

11. The nebulizer according to claim 1, further comprising
a medicine identifying section for reading information of said medicine encoded and attached to a container of said medicine, using either one of optical means or magnetic means.

12. The nebulizer according to claim 11, wherein
said information of said medicine encoded and attached to said medicine container is a bar code.

13. The nebulizer according to claim 1, further comprising
a medicine amount detecting section for detecting amount of said medicine in said medicine container.

14. The nebulizer according to claim 13, wherein
said medicine amount detecting section detects amount of said medicine in said medicine container by optical means.

15. A server, comprising:
a receiving section for receiving information from a nebulizer; and
a storage section for storing said received information; wherein
said nebulizer includes
a treatment section turning medicine into nebulized particles for spraying, a measuring section measuring respiratory function of a patient, and a transmitting section for transmitting at least one of treatment record data from said treatment section, measurement result data from said measuring section, and outer air measurement result data from outer air measuring section measuring at least one of temperature, humidity and atmospheric pressure, said information being transmitted from said transmitting section of said nebulizer to said receiving section of the server.

16. The server according to claim 15, further comprising:
an operating section for performing operating process on said stored information; and
a window creating section for creating window data corresponding to at least one of a patient identifier identifying said patient and a nebulizer identifier identifying said nebulizer based on said stored information and said information performed with operating process.

17. The server according to claim 16, further comprising:
a memory section for storing a password of a user allowed to browse said window;
an authenticating section for authenticating the password referring to said memory section;
an accepting section for accepting a browsing request of said window specifying at least one of said patient identifier and said nebulizer identifier as a condition, and the password of the user requesting browsing of said window, from a processing device with a transmission function; and
a display section for displaying window data corresponding to said specified condition on said processing device when said accepted password is authenticated at said authenticating section.

18. The server according to claim 17, further comprising:
a write accepting section for accepting, from processing device, writing of comment information by a user allowed to browse said display section addressed for a prescribed user; wherein
said display section displays said comment information accepted at said write accepting section on said processing device, when a request for browsing said window of said prescribed user is accepted at said accepting section from processing section.

19. The server according to claim 15, further comprising:
a memory section for storing use period of said nebulizer for each nebulizer identifier; and
a charging section for charging usage fee to said patient using said nebulizer when the utilization period of said nebulizer reaches prescribed period.

20. A nebulizer system, comprising:
a server;
a first processing device connected to a nebulizer;
a second processing device used by a doctor; and
a third processing device used by a related service provider, connected with a network; wherein
said nebulizer includes
a treatment section for turning a medicine into nebulized particles and for spraying the particles,
a measuring section for measuring respiratory function of a patient,
an outer air measuring section for measuring at least one of temperature, humidity and air pressure, and
a transmitting section for transmitting at least one of treatment record data from said treatment section, a measurement result data from said measuring section, and outer air measuring result data from said outer air measuring section;
said server includes
a receiving section for receiving information transmitted from said transmitting section of said nebulizer, and
a storage section for storing said received information; and wherein
said second processing device includes
a transmitting section for transmitting a request for browsing said window specifying said patient identifier as a condition, and a password of said doctor requesting browsing of said window,
a display section displaying said window when said password is authenticated at authenticating section of said server, and
a transmitting section for transmitting a comment for said patient to said first processing device.

21. The nebulizer system according to claim 20, wherein
said third processing device includes
a transmitting section for transmitting a request for browsing said window specifying said nebulizer identifier as a condition, and a password of said related service provider requesting browsing of said window, and
a display section displaying said window when said password is authenticated at authenticating section of said server.

22. The nebulizer system according to claim 20, wherein
said first processing device includes
a transmitting section for transmitting a request for browsing said window of said patient using said nebulizer connecting to the first processing device, and a password of said patient,
a display section displaying said window when said password is authenticated at authenticating section of said server, and
a transmitting section for transmitting a comment for said doctor to said second processing device.

23. The nebulizer system according to claim 20, wherein
said server further includes a charging section for charging usage fee of said nebulizer to said first processing device,
said first processing device further includes a first settling section for settling said usage fee with said server, and
said server further includes a second settling section for settling costs including said usage fee with said third processing device.

24. A method of identifying a medicine in a nebulizer, wherein
information of a medicine encoded and attached to a medicine container is read by at least one of optical means and magnetic means.

25. The method of identifying a medicine in a nebulizer according to claim 24, wherein
said information of the medicine encoded and attached to said medicine container is a bar code.

26. A medicine amount detecting method in a nebulizer, wherein
medicine amount in a medicine container is detected using optical means.

27. A method of managing information in a nebulizer system including a server, a first processing device connected to a nebulizer, a second processing device used by a doctor and a third processing device used by a related service provider, connected with a network; wherein
said nebulizer executes
a treatment step of turning a medicine into nebulized particles and spraying the particles,
an outer air measuring step of measuring at least one of temperature, humidity and atmospheric pressure, and
a transmitting step of transmitting at least one of treatment record data from said treatment step, measurement result data from said measuring step, and outer air measurement result data from said outer air measuring step;
said server executes
a receiving step of receiving information transmitted by said transmitting section of said nebulizer, and
a storing step of storing said received information;
said second processing device executes
a transmitting step of transmitting a request for browsing said window specifying said patient identifier as a condition, and a password of said doctor requesting browsing of said window, and
a displaying step of displaying said window when said password is authenticated at authenticating step of said server, and
a transmitting step of transmitting a comment for said patient to said first processing device.

28. The method of managing information in a nebulizer system according to claim 27, wherein
said third processing device executes
a transmitting step of transmitting a request for browsing said window specifying said nebulizer identifier as a condition, and a password of said related service provider requesting browsing of said window, and
a displaying step of displaying said window when said password is authenticated at authenticating step of said server.

29. The method of managing information in a nebulizer system according to claim 27, wherein
said first processing device executes
a transmitting step of transmitting a request for browsing said window of said patient using said nebulizer connected to the first processing device, and a password of said patient,
a displaying step of displaying said window when said password is authenticated at authenticating step of said server, and
a transmitting step of transmitting a comment for said doctor to said second processing device.

30. The method of managing information in a nebulizer system according to claim 27, wherein
said server further executes a charging step of charging usage fee of said nebulizer to said first processing device,
said first processing device further executes a first settlement step of settling said usage fee with said server in a preset method, and
said server further executes a second settlement step of settling costs including said usage fee with said third processing device.

31. A nebulizer information managing program product causing a computer to execute a method of managing information in a nebulizer system including a server, a first processing device connected to said nebulizer, a second processing device used by a doctor, and a third processing device used by a related service provider, connected with a network;
the program product causing the computer to execute in said nebulizer
a treatment step of turning a medicine into nebulized particles and for spraying the particles,
a measuring step of measuring respiratory function of a patient,
an outer air measuring step of measuring at least one of temperature, humidity and atmospheric pressure, and
a transmitting step of transmitting at least one of treatment record data from said treatment step, measurement result data from said measuring step, and outer air measuring result data from said outer air measuring step;
the program product further causing the computer to execute in said server
a receiving step of receiving information transmitted by said transmitting section of said nebulizer, and
a storing step of storing said received information,
the program product further causing the computer to execute in said second processing device
a transmitting step of transmitting a request for browsing said window specifying said patient identifier as a condition, and a password of said doctor requesting browsing of said window,
a displaying step of displaying said window when said password is authenticated at authenticating step of said server, and
a transmitting step of transmitting a comment for said patient to said first processing device.

32. The nebulizer information managing program product according to claim 31, the program product further causing the computer to execute in said third processing device
a transmitting step of transmitting a request for browsing said window specifying said nebulizer identifier as a condition, and a password of said related service provider requesting browsing of said window, and
a displaying step of displaying said window when said password is authenticated at authenticating step of said server.

33. The nebulizer information managing program product according to claim 31, the program product further causing the computer to execute in said first processing device
a transmitting step of transmitting a request for browsing said window of said patient using said nebulizer connected to the first processing device, and a password of said patient,
a displaying step of displaying said window when said password is authenticated at authenticating step of said server, and
a transmitting step of transmitting a comment for said doctor to said second processing device.

34. The nebulizer information managing program product according to claim 31, the program product further causing the computer to execute in said server a charging step of charging usage fee of said nebulizer to said first processing device,
the program product further causing the computer to execute in said first processing device a first settlement step of settling said usage fee with said server in a preset method, and
the program product further causing the computer to execute in said server a second settlement step of settling costs including said usage fee with said third processing device.
